(19) United States
(12) Patent Application Publication
(10) Pub. No.: US 2017/0049328 A1
(43) Pub. Date: Feb. 23, 2017

(54) RAMAN AND RESONANT RAMAN DETECTION OF VULNERABLE PLAQUE OPTICAL ANALYZER AND IMAGER

(71) Applicants: Robert R. Alfano, Bronx, NY (US); Cheng-hui Liu, Flushing, NY (US); Vidyasagar Sriramoju, Dobs Ferry, NY (US)

(72) Inventors: Robert R. Alfano, Bronx, NY (US); Cheng-hui Liu, Flushing, NY (US); Vidyasagar Sriramoju, Dobs Ferry, NY (US)

(21) Appl. No.: 15/243,607

(22) Filed: Aug. 22, 2016

Related U.S. Application Data
(60) Provisional application No. 62/283,108, filed on Aug. 21, 2015.

(51) Int. Cl. A61B 5/00; A61B 5/02

(52) U.S. Cl. CPC A61B 5/0086; A61B 5/0075; A61B 5/02007; A61B 2562/0238

(57) ABSTRACT

Vulnerable plaque (VP) is the main cause of death from heart attacks. All currently available methods developed to diagnose VP lack sensitivity and or specificity and are still unable to identify VP. Our patent addresses the problem to diagnose VP in arteries. The teachings here disclose a vulnerable plaque optical analyzer (VPOA) and Imager (VOPAI) for monitoring arterial walls by measuring whether the fingerprint Raman spectrum of adipose (lipid) tissue using Resonance Raman (RR) and common Raman(R) signals of aortic intimal wall layer. The RR and R lines of lipid determine presentation of VP.

### BACKGROUND OF THE INVENTION

1. Field of the Invention

The invention generally relates to detection of vulnerable plaque and, more specifically, to a Raman and Resonant Raman Detection of Vulnerable Plaque Optical Analyzer and Imager.

2. Description of the Prior Art

In 2010, over 595,000 deaths were due to heart diseases. Seventy percent of acute coronary events are fatal. These deaths result from the sudden disruption of a particular type of athermanous plaque called thin cap fibroatheroma (TCFA) or vulnerable plaque (VP), see link to the video https://www.youtube.com/watch?v=wHQY0o8RdS4. It is, therefore, essential to identify the individual at risk. No test today allows for the precise localization and identification of VP.

Several non-invasive and invasive imaging techniques have been developed to study the vessel wall in detail. Most diagnostic devices are or have been designed for the purpose of detecting VPs. Coronary angiography, high resolution magnetic resonance imaging. CT scan, nuclear imaging and minimally invasive endoscopic imaging procedures such as intravascular ultrasound and optical coherence tomography lack sensitivity and or specificity and are still unable to reliably identify VP regions.

### SUMMARY OF THE INVENTION

NIR and visible Raman spectroscopy offers a potential way to find VP. Raman spectra have been used to assess coronary plaque composition [1, 2]. The visible source below 620 nm are for RR and NIR sources above 620 nm say 785 nm are for R.

Raman signals are based on a shift of photons to a different wavelength due to the tissue structure and composition. Raman spectroscopy is capable of differentiating atherosclerotic plaque from diffuse intima thickening. This result indicates that Raman spectroscopy has the potential to accomplish plaque localization and characterization with a precision of <65 micron.

The resonance effect in resonance Raman (RR) spectroscopy occurs when the energy of the excitation laser is adjusted such that it and/or the energy of the scattered photons approaches the energy of an electronic transition of the chemical bonds in a molecule to an excited state. As the energy of the excitation approaches an optical transition energy level, the vibrational resonance effect occurs that greatly enhances the scattering and, thus, the peak intensities in the Raman spectra increase by as much as 1000 fold. The peaks from non-resonance-enhanced molecules seemingly disappear under the intensity of the resonance-enhanced spectral peaks. Chromophores, and other large conjugated molecules, experience stretching and bending vibrations that can be enhanced by the excitation laser and the RR spectra collected from them exhibit enhanced peaks. In artery wall cells and tissues containing so many large biomolecules with multiple vibrations, the many advantages of RR spectroscopy for biomedical diagnosis over conventional Raman include: the spectra collected from resonance enhanced molecules can be detected at low molecular concentrations, and the activity of particular molecular species can be targeted preferentially. Specific biomolecules in the cell and organelles contain fluorophores, such as flavins, NADH, lipids, collagens, elastin, carotenoid and the heme proteins, such as the mitochondrial cytochromes. These are coupled to vibrations which can be enhanced by RR. to be larger than spectral fluorescence wing observed in R using NIR 785 nm and 632 nm lasers. Below 600 nm, such as at 532 and 288 nm, RR appears. It is best to use a laser with 532 nm since flavins in its tail are the coupler molecules to enhance the vibrations by >100×.

The RR and R Raman lines due to lipids are key metrics to detect the presence of VP in arteries in situ for in vivo applications. RR and R Raman signals from the artery walls are spectral flat in certain spectral Raman shills while those from lipids are sharp and intense. This disclosure teaches the key location to measure VP via RR and R. The thickness of an intimal wall layer compared to lipids determines the degree of VP. A strong Raman lipid signal indicates thin artery walls and the existence of vulnerable plaques. The salient fingerprint Raman vibration frequencies for VP detection are 1435 cm⁻¹, 2850 cm⁻¹ and 2892 cm⁻¹ for RR. and R Hard calcified plaque has a very strong Raman fingerprint at 957 cm⁻¹. FIGS. 1 and 2 show the key teachings to detect VP.

### BRIEF DESCRIPTION OF THE DRAWINGS

The above and other aspects, features and advantages of the present invention will be more apparent from the following description when taken in conjunction with the accompanying drawings, in which:

FIG. 1 shows Raman spectra of Aorta intimal wall tissue, fat tissue from aorta wall, and cholesterol powder.

FIG. 2 shows the intensity changes of Raman spectra in region 1250 cm⁻¹ to 1700 cm⁻¹ for different wall thickness of model VP.

FIG. 3 is a schematic of a Raman Ratio meter for VP.

### DETAILED DESCRIPTION

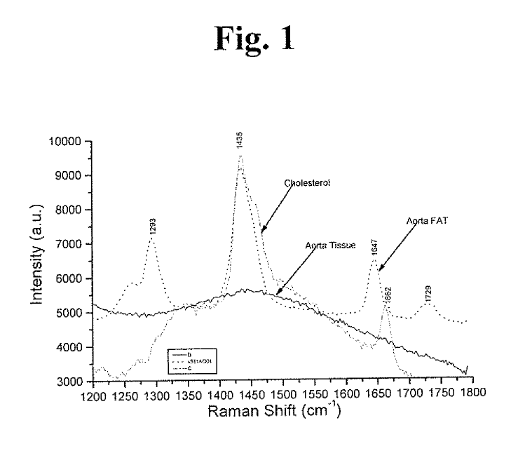

FIG. 1 shows normalized to Baseline 1341 cm−1 of Raman spectra of Aorta intimal wall tissue, FAT tissue is from aorta adventitial wall, and Cholesterol powder (Sigma corp.), exposure time is 5 seconds, excitation wavelength at 633 nm, Scan Center at 680 nm.

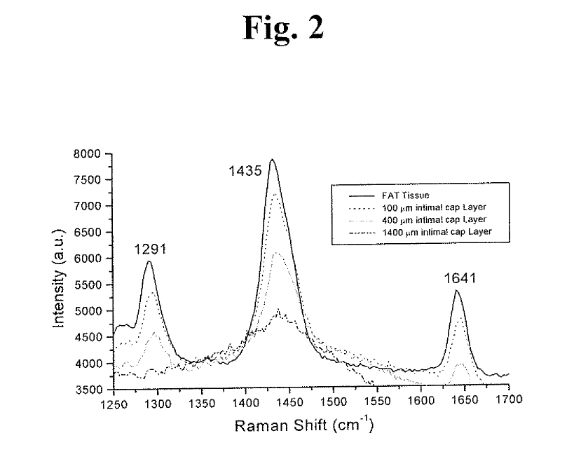

FIG. 2 shows the intensity changes of Raman spectra in region 1250 cm⁻¹ to 1700 cm⁻¹ (scan center 680 nm, (mode of 1435 cm⁻¹) versus thickness of intimal layers on the top of fat tissue.

The VPOA fiber-based unit will be used to determine the presence and location of vulnerable plaques. The health risk to patients from the VPOA fiber unit is similar to that of commonly existing fiber probes entering arteries and heart.

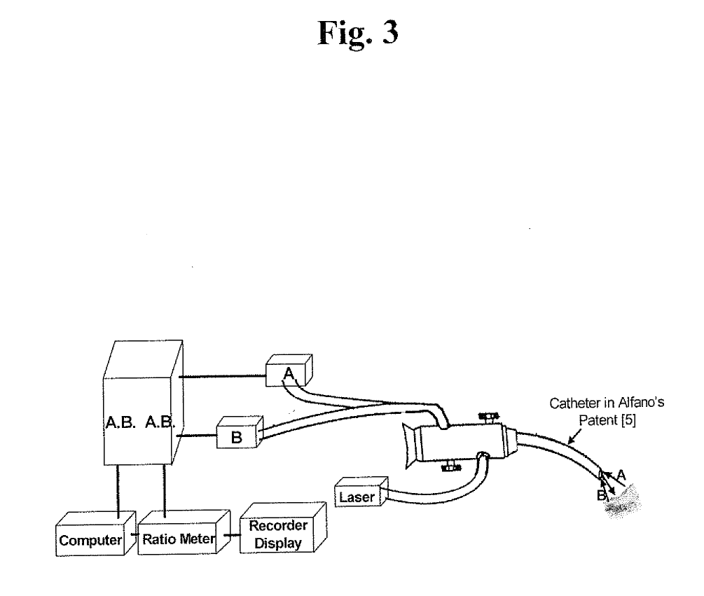

Phase project was focused on designing thickness algorithm and building of the VPOA prototype to be tested on arteries from dogs. Schematic diagram is shown in FIG. 3. A Filter probe enters inside an artery, Raman Signal A is from Arterial Tissue, Raman Signal B is from Calcified Plaque or VP, Fibers A and B with notch filters and a set of narrow band filter selected At Raman peaks of 957 cm⁻¹, 1293 cm⁻¹, 1435 cm⁻¹, 1647 cm⁻¹, 2850 cm⁻¹ and 2892 cm⁻¹ send signal to photodetectors shown as box A and box B; laser is with narrow band filter, The Box (A.B.,A.B.) is electronic converter, the ratio is equal Raman peak intensity IB to background intensity IA (IB/IA) on computer screen shows Ratio or Spectrum.

Raman spectra was recorded using VPAO in arteries ex vivo of several animals and humans. Rotation of fiber will be used to obtain Raman cross-sectional images along the artery.

Optical spectroscopy methods such as Raman spectroscopy (RS) and fluorescence (FL) spectroscopy have widely been used to diagnose artery diseases since the late 1980's. None have been used to diagnose VP. Since fluorescence spectra of tissue involve emissions from various molecules and are usually broad, it is difficult to use FL spectra to distinguish contributions from each of the involved molecules. Raman spectra provide narrow spectral features that can be related to the specific molecular structure even for complex multi-component samples such as biological tissue. The detailed biological information obtained from Raman spectra is suitable for histo-chemical analysis of the artery tissue. It has been reported that Raman spectroscopy, as a minimally invasive and non-destructive optical technique, can provide histo-chemical information at the molecular level on the contents of cholesterol and calcification in atherosclerotic plaque.

As a proof of concept, Raman spectra from arterial tissue samples were studied. Development of the VPOAI will be based on the results on RR and R Raman study using a fiber with prism at tip which is rotated about for 360 degrees and the fiber is translated to move along the artery to acquire a 3D Raman image to locate the VP spatial in arteries.

The sample structure was prepared by placing a variable number of the aorta intimal wall tissue layers on top of adipose tissue to vary the total thickness of the layer from 50 to 2,000 μm. Raman spectra of adipose tissue and tissue were obtained and the intensity changes of the Raman modes were measured versus thickness of the aorta intimal wall tissue layers. Principal characteristic fingerprint Raman vibration modes from adipose tissue were found at 1435 cm⁻¹, 2850 cm⁻¹ and 2892 cm⁻¹. The intensities of the modes of 2850 cm⁻¹ and 2892 cm⁻¹ are about four-times stronger than that of the 1435 cm⁻¹ mode. When the thickness of the cap intimal wall tissue over fat is thin, the fat signal is strong and detects the VP region. This has potential applications for artery disease screening and clinical diagnosis. The combination of RR and R Raman is superior to any cardiology technique to diagnose VP in vivo.

Two laser wavelengths can be used separated from each other by the vibrational frequency. One is called the pump laser and the other is called the Raman laser. For example, using a pump wavelength of 532 nm and a Raman wavelength of 627 nm this results in a difference of 2850 cm⁻¹ which matches the vibrational frequency of at least one of the molecules. The Raman spectra will be enhanced by the transfer of energy from the pump laser to the Raman beam. This enhanced signal enhances the imaging of an artery and detection of VP.

VP is the most dangerous plaque, because it could rupture clogging the artery and causing death (70% of the 500000) are from VP. The VP regions are found by lipid RR and R signals. The thickness of intimal wall layer over lipids determines existence of VP. A strong Raman signal of lipids indicates thin wall of artery and vulnerable plaques. The Raman vibration modes for VP are strong bands at 1435 cm⁻¹, 2850 cm⁻¹ and in 2892 cm⁻¹. The VPOAI unit will be used to determine the existence and regions of vulnerable plaques in RR and R images.

While the invention has been shown and described with reference to certain embodiments thereof, it will be understood by those skilled in the art that various changes in form and detail may be made therein without departing from the spirit and scope of the invention as defined by the appended claims and their equivalents.

1. Method of detecting vulnerable plaque comprising the steps of inserting a filtered fiber probe into an artery proximate to arterial tissue; applying a laser excitation light in a range of 450 nm to 600 nm; developing an image of the artery wall using RR or R for lipids and plaque; generating Raman signal A from arterial tissue; generating Raman signal B from calcified plaque or VP; filtering Raman signals A and B to transmit Raman peaks at 957 cm⁻¹; detecting at least one Raman background peak signal A and at least one Raman peak signal B; establishing ratios I_{B}/I_{A} to detect VP and plaques.

2. A method as defined in claim 1, wherein laser excitation is performed at 532 nm and 785 nm in tissue arteries.

3. A method as defined in claim 1, wherein a filtered optical fibers bundle of mm size is used to diagnose in vivo of tissue and VP.

4. A method as defined in claim 1, wherein other lasers in blue-green range are used to excite chromophores for vibration enhancement of Raman for RR such as Argon, diode, semiconductor lasers, SHG of YAG, HeCd.

5. A method as defined in claim 1, wherein Raman lines are detected at about 1435 cm⁻¹ and/or 2850 cm⁻¹.

6. A method as defined in claim 1, wherein two or more of the Raman lines are used to detect VP regions and Lipid regions.

7. A method as defined in claim 1, wherein two laser wavelengths are used separated from each other by the vibrational frequency of a molecule under investigation.

8. A method as defined in claim 7, wherein one wavelength is generated by a pump laser and the other wavelength is generated by a Raman laser.

9. A method as defined in claim 8, wherein enhanced Raman signal gain is generated at vibrations for imaging areas/regions of plaque and VP spatially in the wall of an artery.
